(19) United States
(12) Patent Application Publication
(10) Pub. No.: US 2026/0193628 A1
(43) Pub. Date: Jul. 9, 2026

(54) SALT ACTIVE NUCLEASE COMPOSITIONS AND METHODS

(71) Applicant: New England Biolabs, Inc., Ipswich, MA (US)

(72) Inventors: Carine Robichon-Iyer, Cambridge, MA (US); Minyong Chen, Ipswich, MA (US); Laura Ott, Beverly, MA (US)

(73) Assignee: New England Biolabs, Inc., Ipswich, MA (US)

(21) Appl. No.: 19/190,213

(22) Filed: Apr. 25, 2025

Related U.S. Application Data
(60) Provisional application No. 63/639,305, filed on Apr. 26, 2024.
(60) Provisional application No. 63/640,498, filed on Apr. 30, 2024.

(51) Int. Cl. C12N 9/22; C12N 9/10; C12N 11/00

(52) U.S. Cl. CPC C12N 9/22; C12N 9/1007; C12N 11/00; C07K 2319/21; C07K 2319/24; C07K 2319/31; C07K 2319/80; C12Y 201/01063; C12Y 301/21001

(57) ABSTRACT

The present disclosure relates, according to some embodiments, to systems, apparatus, compositions, methods, and workflows that include salt active nucleases (e.g., salt active nuclease variants) with desirable properties including, for example, salt tolerance.

### CROSS-REFERENCE TO RELATED APPLICATIONS

This application claims priority to U.S. Provisional Application No. 63/639,305 filed Apr. 26, 2024 and U.S. Provisional Application No. 63/640,498 filed Apr. 30, 2024. The contents of all of the above are hereby incorporated in their entirety by reference.

### SEQUENCE LISTING STATEMENT

This disclosure includes a Sequence Listing submitted electronically in .xml format under the file name “NEB-501.xml” created on Apr. 26, 2024, and having a size of 15,813 bytes. This Sequence Listing is incorporated herein in its entirety by this reference.

### BACKGROUND

Many purification methods involve one or more steps that include high salt conditions. High salt conditions may be desirable, for example, to stabilize one or more molecules of interest, to control its interactions (e.g., drive its association or dissociation) with one or more components present in the composition in which it exists, and/or to support desirable conditions for cell lysis. However, high salt conditions may limit or prevent activity of enzymes used to remove contaminants such as polynucleotides during a purification of a protein, metabolite, virus, cell or other material of interest.

### SUMMARY

Accordingly, needs have arisen for improved nucleases that are active in the presence of salts. The present disclosure provides salt active nucleases (including salt active nuclease variants) and related methods, kits, and compositions. In some embodiments, a salt active nuclease variant may have an amino acid sequence that is ≥90% (e.g., ≥92%, ≥94%, ≥95%, ≥96%, ≥98%, ≥99%) identical to a reference salt active nuclease (e.g., one or more of SEQ ID NOS:1-4), having at its position corresponding to position 52 of SEQ ID NO:1 an amino acid other than Q (glutamine) (e.g., may be S52 (serine52)), and having catalytic activity as an endonuclease and/or an endoribonuclease at a total salt concentration of 250 mM to 1 M. In some embodiments, a salt active nuclease variant may have an amino acid sequence that is identical to SEQ ID NO:2 or identical to SEQ ID NO:3. A salt active nuclease variant identical to SEQ ID NO:3 may have, for example, a signal peptide, a purification tag, or a linker at X1 and/or X215. For example, a X1 may be a signal peptide, a purification tag, or a linker and X215 may be any amino acid or absent, or (b) X1 may be any amino acid or absent and X215 may be a purification tag or a linker or X215. In a salt active nuclease variant having less than 100% identity to a reference sequence, each non-identical position may constitute a substitution relative to the reference. Salt active nuclease variants having less than 100% identity (e.g., with each non-identical position constituting a substitution) to a reference sequence may comprise conservative (e.g., exclusively conservative) substitutions relative to the reference and/or non-conservative (e.g., zero, one, two, three, four, figure, six, or more non-conservative substitutions). According to some embodiments, a salt active nuclease variant may comprise an amino acid sequence having at least 98% identical to SEQ ID NO:2 and wherein X47-X51 and X53-X56 are each, independently, any amino acid. According to some embodiments, a salt active nuclease variant may comprise an amino acid sequence having at least 98% identical to SEQ ID NO:2 and wherein X47, X49-X51, and X53-X55 each, independently, may be C, D, E, H, K, N, P, Q, R, S, T, or Y, and X48 and X56 are each, independently, A, L, I, M, W, F, C, G, or P.

A salt active nuclease variant, according to some embodiments, may have ≥30% (e.g., ≥35%, ≥40%, ≥45%, ≥50%, ≥55%, ≥60%) of its peak catalytic activity in the presence of a total salt concentration of 250 mM to 1 M (e.g., 250 mM to 750 mM, 300 mM to 600 mM), the salt consisting essentially of NaCl, KCl, or NaCl and KCl. In some embodiments, a salt active nuclease variant may have ≥30% (e.g., ≥35%, ≥40%, ≥45%, ≥50%, ≥55%, ≥60%) of its peak catalytic activity in the presence of a total salt concentration of 50 mM to 150 mM (e.g., 75 mM to 125 mM), the salt consisting essentially of MgCl₂, Mg(NO₃)₂, or MgCl₂ and Mg(NO₃)₂.

A salt active nuclease variant may have, in some embodiments, ≥60% (e.g., ≥65%, ≥70%, ≥75%, ≥80%) of its peak catalytic activity at pH ≥8. According to some embodiments, a salt active nuclease may have ≥60% (e.g., ≥65%, ≥70%, ≥75%, ≥80%) of its peak catalytic activity at 40° C.-55° C. A salt active nuclease may be an immobilized salt active nuclease variant comprising the salt active nuclease variant and a solid support (e.g., a magnetic bead, an agarose bead, a polystyrene bead, a polyacrylamide bead or a chitin bead).

The present disclosure also provides compositions including salt active nucleases. For example, compositions may include a salt active nuclease variant and one or more salts (e.g., a total of 50 mM-150 mM of MgCl₂, Mg(NO₃)₂, or MgCl₂ and Mg(NO₃)₂; a total of 250 mM-1 M of NaCl, KCl, or NaCl and KCl). In some embodiments, a composition may comprise a buffering agent and/or optionally one or more other materials of interest (e.g., proteins, polysaccharides, cells, phage particles, viral particles, liposomes, and/or micelles). Salt active nucleases may be included in fusions. For example, a fusion protein may comprise a single polypeptide chain, the single peptide chain comprising (a) a salt active nuclease variant and (b) a SSO7d DNA binding peptide, a transcription factor, an antibody, protein A, a maltose binding domain, a histidine tag, a chitin binding domain, an alpha mating factor, an O⁶-alkylguanine-DNA alkyltransferase, and/or albumin. The present disclosure further provides, in some embodiments, kits for hydrolyzing DNA and/or RNA. A kit may comprise, for example, (a) any of the salt active nuclease variants disclosed herein, and (b) a buffering agent.

The present disclosure also provides methods including salt active nucleases. A method may comprise, for example, hydrolyzing DNA and RNA. In some embodiments, a method for hydrolyzing DNA and RNA may comprise contacting (a) a composition comprising DNA and RNA, and (b) a salt active nuclease variant to form a reaction mixture comprising DNA hydrolysis products and RNA hydrolysis products. A reaction mix may have any desired pH (e.g., 6.5-12, 8-10) and/or any desired temperature (e.g., 2° C.-60° C., 40° C.-55° C.). In some embodiments, a composition comprising DNA and RNA and/or a reaction mixture may further comprise (a) a total salt concentration of 250 mM to 1 M, the salt consisting essentially of (i) NaCl, (ii) KCl, or (iii) NaCl and KCl or (b) a total salt concentration of 50 mM to 150 mM, the salt consisting essentially of (i) MgCl₂, (ii) Mg(NO₃)₂, or (iii) MgCl₂ and Mg(NO₃)₂. In some embodiments, the reaction mixture comprises ≤10% of the DNA that was in the composition comprising DNA and RNA (prior to contact with the nuclease) and/or ≤10% of the RNA that was in the composition comprising DNA and RNA (prior to contact with the nuclease). A method may further comprise detecting the amount of DNA and/or RNA is present in the composition comprising DNA and/or RNA and/or may further comprise detecting the amount of DNA and/or RNA is present in the reaction mixture. In some embodiments, a composition comprising DNA and RNA may further comprise one or more DNA-binding proteins (e.g., histones). For example, a composition comprising DNA and RNA may further comprise one or more nucleosomes. In some embodiments, a method may include isolating and/or purifying a material of interest from the composition and/or the reaction mixture.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIGS. 1A, 1B, and 1C show nuclease activity of example salt active nucleases vsEndA and vsEndA-Q52S on dsDNA (FIG. 1A), ssDNA (FIG. 1B) and RNA (FIG. 1C) substrates at various NaCl concentrations.

FIG. 2 shows nuclease activity of an example salt active nuclease vsEndA-Q52S in various NaCl concentrations compared to 2 commercially available endonucleases.

FIG. 3 shows nuclease activity of example salt active nucleases vsEndA and vsEndA-Q52S on dsDNA across a range of pHs.

FIG. 4 shows nuclease activity of example salt active nucleases vsEndA and vsEndA-Q52S on dsDNA across a range of temperatures.

FIG. 5 shows nuclease activity of example salt active nucleases vsEndA (left gel) and vsEndA-Q52S (right gel) on lambda DNA at pH 8.5 and 500 mM NaCl.

FIG. 6 shows nuclease activity of example salt active nucleases vsEndA (left gel) and vsEndA-Q52S (right gel) on a total RNA extract from HEK cells at pH 8.5 and 500 mM NaCl.

FIG. 7 shows nuclease activity of example salt active nucleases vsEndA (left gel) and vsEndA-Q52S (right gel) on a fluorescent RNA substrate at pH 8.5 and 500 mM NaCl.

FIGS. 8A, 8B, and 8C show nuclease activity of example salt active nucleases vsEndA and vsEndA-Q52S in the presence of KCl (FIG. 8A), MgCl₂ (FIG. 8B), and Mg(NO₃)₂ (FIG. 8C).

FIG. 9 shows nuclease activity of an example salt active nuclease vsEndA-Q52S and 2 commercially available endonucleases on calf thymus DNA at 500 mM NaCl pH 8.5 at 25° C. (upper gel) and 4° C. (lower gel).

FIGS. 10A and 10B shows an example embodiment of linear release of fluorescence (RFU as Relative Unit of Fluorescence) from dsDNA (FIG. 10A) or ssDNA (FIG. 10B) substrates relative to the concentration of both enzymes.

FIG. 11 shows nuclease activity of an example salt active nuclease vsEndA-Q52S and a commercially available endonuclease (Supplier B) on mononucleosomal DNA at 100 mM, 250 mM, and 500 mM NaCl.

### BRIEF DESCRIPTION OF THE SEQUENCES

Some embodiments of this disclosure relate to the following provided sequences of example polynucleotides and/or example polypeptides.

SEQ ID NO:1 is an example salt active nuclease variant.

APPSSFSKAKKEAVKIYLDYPTSFYCGCDITWKNKKKGIPELESCGYQVR
KSEKRASRIEWEHVVPAWQFGHQRQCWQKGGRKNCTRNDKQFKSMEADLH
NLVPAIGEVNGDRSNFRFSQWNGSKGAFYGQCAFKVDFKGRVAEPPAQSR
GAIARTYLYMNNEYKENLSKAQRQLMEAWNKQYPVSTWECTRDERIAKIQ
GNHNQFVYKACTK

SEQ ID NO:2 is an example salt active nuclease variant, wherein X47, X49-X51, and X53-X55 independently may be any amino acid (e.g., one of C, D, E, H, K, N, P, Q, R, S, T, and Y), X52 is any amino acid other than Q (e.g., C, D, E, H, K, P, R, S, T or Y) and X48 and X56 may be any amino acid (e.g., one of A, L, I, M, W, F, C, G, and P). The underlined amino acids may be spatially close to X52 and amenable to conservative (or non-conservative) substitution to improve cold activity, salt activity, salt tolerance, and/or salt indifference.

APPSSFSKAKKEAVKIYLDYPTSFYCGCDITWKNKKKGIPELESCGYXXX
XXXXXXSRIEWEHVVPAWQFGHQRQCWQKGGRKNCTRNDKOFKSMEADLH
NLVPAIGEVNGDRSNFRFSQWNGSKGAFYGQCAFKVDFKGRVAEPPAQSR
GAIARTYLYMNNEYKENLSKAQRQLMEAWNKQYPVSTWECTRDERIAKIQ
GNHNQFVYKACTK

SEQ ID NO:3 is an example salt active nuclease variant, wherein X1 may be a purification tag, a linker, any amino acid or may be absent, X53 is any amino acid other than Q (e.g., C, D, E, H, K, N, P, R, S, T or Y), and X215 may be a signal peptide, a purification tag, a linker, any amino acid or may be absent.

XAPPSSFSKAKKEAVKIYLDYPTSFYCGCDITWKNKKKGIPELESCGYQV
RKXEKRASRIEWEHVVPAWQFGHQRQCWQKGGRKNCTRNDKQFKSMEADL
HNLVPAIGEVNGDRSNFRFSQWNGSKGAFYGQCAFKVDFKGRVAEPPAQS
RGAIARTYLYMNNEYKENLSKAQRQLMEAWNKQYPVSTWECTRDERIAKI
QGNHNQFVYKACTKX

SEQ ID NO:4 is an example salt active nuclease variant, wherein a cleaved signal peptide is underlined.

MKLIRLVISLIAVSFTVNVMAAPPSSFSKAKKEAVKIYLDYPTSFYCGCD
ITWKNKKKGIPELESCGYQVRKSEKRASRIEWEHVVPAWQFGHQRQCWQK
GGRKNCTRNDKQFKSMEADLHNLVPAIGEVNGDRSNFRFSQWNGSKGAFY
GQCAFKVDFKGRVAEPPAQSRGAIARTYLYMNNEYKFNLSKAQRQLMEAW
NKQYPVSTWECTRDERIAKIQGNHNQFVYKACTK

SEQ ID NO:5 is an example salt active nuclease (vsEndA; Q2XSL7), wherein a (cleavable) 21-amino acid signal peptide is underlined.

MKLIRLVISLIAVSFTVNVMAAPPSSFSKAKKEAVKIYLDYPTSFYCGCD
ITWKNKKKGIPELESCGYQVRKQEKRASRIEWEHVVPAWQFGHORQCWQK
GGRKNCTRNDKQFKSMEADLHNLVPAIGEVNGDRSNFRFSQWNGSKGAFY
GQCAFKVDFKGRVAEPPAQSRGAIARTYLYMNNEYKFNLSKAQRQLMEAW
NKQYPVSTWECTRDERIAKIQGNHNQFVYKACTK

SEQ ID NO:6 is an example salt active nuclease (VvnI)

APPSSFSAAKQQAVKIYQDHPISFYCGCDIEWQG_KKGIPNLETCGYQVR
KQQTRASRIEWEHVVPAWQFGHHRQCWQKGGRKNCSKNDQQFRLMEADLH
NLTPAIGEVNGDRSNFNFSQWNGVDGVSYGRCEMQVNFKQRKVMPQTELR
GSIARTYLYMSQEYGFQLSKQQQQLMQAWNKSYPVDEWECTRDDRIAKIQ
GNHNPFVQQSCQTQ

SEQ ID NO:7 is an example hairpin dsDNA substrate comprising 5′ FAM labeled and 3′ black hole quencher (“BHQ1”) labeled ends. When the probe is heated to 95° C. and then gradually cooled, it folds into a hairpin structure in which the quencher quenches the fluorophore. Cleavage of the DNA hairpin by a nuclease (e.g., a SAN) releases FAM, resulting in an increase in fluorescence that can be measured at 517 nm using a spectrophotometer.

56-FAM-TCTAAGCCGTGTACATTTTTTGTACACGGCTTAGA-BHQ1-3′

SEQ ID NO:8 is an example linear ssDNA substrate comprising 5′ FAM and 3′ BHQ1 labeled ends.

56-FAM-TGAAGTAATCTGTTA-BHQ1-3′

SEQ ID NO:9 is an example linear RNA substrate optionally comprising a FAM-labeled 5′ end.

56-FAM-AAGGAGAAGAGAAGAGGAAGAAAACUAACACAGGAGAGAGAA
GGA

SEQ ID NO:10 is an example DNA sequence encoding an example salt active nuclease.

GCACCACCAAGCAGTTTTTCTAAAGCTAAGAAAGAGGCTGTGAAAATTTA
CCTGGACTACCCTACTAGTTTTTACTGTGGTTGTGATATTACATGGAAAA
ATAAAAAGAAAGGAATTCCGGAGTTAGAGTCATGTGGGTACCAGGTCCGT
AAAAGCGAAAAGCGTGCGAGCCGTATCGAGTGGGAACACGTCGTACCTGC
ATGGCAATTTGGACATCAGCGTCAATGCTGGCAGAAAGGGGGACGCAAAA
ATTGCACTCGCAACGATAAACAATTCAAAAGCATGGAGGCAGATTTGCAT
AACCTTGTACCTGCCATCGGCGAGGTGAATGGAGATCGCTCTAACTTCCG
CTTTTCGCAGTGGAATGGGTCAAAGGGGGCGTTCTATGGGCAATGCGCCT
TCAAGGTAGATTTCAAAGGTCGCGTTGCGGAGCCACCAGCCCAGTCCCGT
GGGGCCATTGCCCGCACTTATTTATACATGAATAACGAGTATAAGTTTAA
TTTGAGCAAAGCCCAGCGTCAACTTATGGAAGCCTGGAATAAACAATATC
CGGTGAGCACCTGGGAGTGCACTCGTGATGAGCGTATTGCCAAAATCCAG
GGGAATCATAACCAATTCGTCTATAAGGCGTGCACTAAGTGA

### DETAILED DESCRIPTION

The present disclosure relates, in some embodiments, to systems, apparatus, compositions, and/or methods for cleaving polynucleotides in the presence of salt (e.g., ≥250 mM salt). For example, the present disclosure provides systems, apparatus, compositions, methods, and workflows that include salt active nucleases with desirable properties including, for example, salt tolerance. A composition may comprise, in some embodiments, a salt active nuclease. For example, a composition may include a catalytically active salt active nuclease comprising an amino acid sequence having ≥85%, ≥90%, ≥92%, ≥94%, ≥95%, ≥96%, ≥98%, or ≥99% identity to SEQ ID NO:1 and ≥50 mM, ≥100 mM, ≥150 mM, ≥200 mM, ≥250 mM, ≥300 mM, ≥350 mM, ≥400 mM, ≥450 mM, ≥500 mM, ≥600 mM, ≥700 mM, ≥800 mM, or ≥900 mM salt (e.g., NaCl).

### General Considerations

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Lists of example species within a particular genus may vary in length at different places throughout the disclosure. Species lists shortened for convenience shall not be construed to exclude example species listed elsewhere in the specification. Any recited method can be carried out in the order of events recited or in any other order which is logically possible.

Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified. Percent ranges with only one end point (e.g., ≥90% or ≤10%) optionally include a second endpoint at the maximum or minimum percentage (e.g., ≥90% includes a range of 90%-100% and ≤10% includes a range of 0%-10%). Ranges (including percent ranges) with only one end point (e.g., ≥90 or ≤10) optionally include a second endpoint 10% higher or 10% lower than the provided endpoint (e.g., ≥90 includes a range of 90-99 and ≤10 includes a range of 1-10). Concentration percentages are w/v percentages unless otherwise indicated.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

As used herein, “catalytically active” refers to the property of a molecule (e.g., a proteinaceous molecule or macromolecule) to function as a catalyst of one or more chemical reactions relative to one or more substrates and products. A catalytically active salt active nuclease or salt active nuclease variant, for example, hydrolyzes one or more polydeoxyribonuceic acids to yield (however briefly) products comprising at least one 5′-phosphorylated oligonucleotide. Catalytic activity of salt active nuclease and/or salt active nuclease variants may be assessed using existing techniques applied to one or more model substrates and/or one or more substrates of interest. For example, effective assays for catalytic activity of salt active nuclease may include size fractionation of products (e.g., on gels or other matrices), radioactive assays, and fluorometric assays (e.g., #234056, #abab252898, AbCam PLC, Cambridge, U.K.; PicoGreen, Nucl. Acids Res. 2003 31(18):e111)). Catalytic activity may be assessed with respect to loss of original DNA (e.g., percent of original DNA remaining), concentration of DNA (e.g., above 10 nts, above 8 nts, above 6 nts, above, above 3 nts in length, 4 nts, or above 2 nts in length), and/or metrics that serve as a proxy thereof.

Peak catalytic activity or peak activity is the highest observed catalytic activity in reactions representing the full range of total salt concentrations, but otherwise maintained under the same conditions (e.g., buffer, temperature, reaction time, type and quantity of substrate, quantity of enzyme, pH). For example, to assess peak catalytic activity empirically, a salt tolerant nuclease variant may be divided into a plurality of aliquots. Each aliquot may be combined with a buffering agent, a test substrate (e.g., SEQ ID NOS:7-9), and differing concentrations of salt (e.g., 0 mM, 250 mM, 500 mM, 750 mM, and 1 M), incubated for a selected time at a selected temperature, and assayed for nuclease activity by any method provided herein or otherwise available. Peak activity for the salt tolerant nuclease variant assayed would be the highest observed activity among the tested concentrations (e.g., 0 mM, 250 mM, 500 mM, 750 mM, and 1 M). The activity at the other salt concentrations may be expressed as a percentage of the peak activity.

In the context of the present disclosure, “container” refers to a human-made container. A container may comprise one or more walls (e.g., defining an interior volume) and optionally one or more openings. Containers comprising one or more openings may further comprise one or more closures (e.g., removable closures) for some or all such openings. A closure optionally may comprise an aperture or a septum, for example, to provide fluid communication with a volume of the container and a connected or inserted tube or syringe. Examples of containers include boxes, cartons, bottles, tubes (e.g., test tubes, microcentrifuge tubes), plates (e.g., 96-well, 384-well plates), vials, pipette tips, and ampules. Containers and/or closures may comprise any desired material including paper, plastics, glass, silicone, composites, metals, alloys, or combinations thereof. Containers and/or closures may comprise materials that are compostable, recyclable, and/or sustainable.

In the context of the present disclosure and with respect to an amino acid residue or a nucleotide base position, “corresponding to” refers to positions that lie across from one another when sequences are aligned, e.g., by the BLAST algorithm. An amino acid position in a functional or structural motif in one endonuclease may correspond to a position within a functionally equivalent functional or structural motif in another endonuclease.

With respect to polynucleic acid, “digest,” as used herein, refers to hydrolyzing or otherwise reducing the size of such polynucleic acid. Unless qualified, digesting a polynucleic acid (e.g., DNA or RNA) includes all degrees of hydrolyzing or otherwise reducing the size of such polynucleic acid, partially up to and including fully. Sites of hydrolysis may be regarded as independent of the nucleotide sequence (“non-specific”), even if some sequence bias is observed under some conditions.

In the context of the present disclosure, “fusion” refers to two or more polypeptides, subunits, or proteins covalently joined to one another (e.g., by a peptide bond). For example, a protein fusion may refer to a non-naturally occurring polypeptide comprising a protein of interest covalently joined to a second polypeptide. Examples of a second polypeptide include a reporter protein (e.g., a green fluorescent protein), a purification tag (e.g., a 6×His or 8×His tag), and expression tag, a polynucleotide binding protein, an enzyme, a conjugation tag (e.g., a SNAP® tag), and a peptide linker (e.g., a flexible linker, an inflexible linker, a cleavable linker). Unless otherwise disclosed, the protein of interest may be nearer to the N-terminal end or nearer to the C-terminal end than the second polypeptide to which it is joined. A fusion protein may have one or more heterologous domains added to the N-terminus, C-terminus, and or the middle portion of the protein. A fusion may comprise a non-naturally occurring combined polypeptide chain comprising two proteins or two protein domains joined directly to each other by a peptide bond or joined through a peptide linker. If two parts of a fusion protein are “heterologous”, they are not part of the same protein in its natural state. In some embodiments, a fusion may comprise a variant salt active nuclease covalently joined to a second polypeptide. In some embodiments, a variant salt active nuclease may include a fusion to an exogenous DNA binding domain, examples of which are provided in Table 1 of U.S. U.S. Pat. No. 11,259,184. Examples of fusion proteins include a salt active nuclease variant fused to an SSO7d DNA binding peptide (see for example, U.S. Pat. No. 6,627,424), a transcription factor (see for example, U.S. Pat. No. 10,041,051), an antibody, protein A (e.g., SpA), a binding domain suitable for immobilization such as maltose binding domain (MBP), a histidine tag (“His-tag”), chitin binding domain, alpha mating factor or an O⁶-alkylguanine-DNA alkyltransferase (e.g., SNAP-Tag®, New England Biolabs, Ipswich, MA (see for example U.S. Pat. Nos. 7,939,284 and 7,888,090)), and/or albumin. The binding peptide may be used to improve solubility or yield of the salt active nuclease variant during the production of the protein reagent. Other examples of fusion proteins include fusions of a salt active nuclease and a heterologous targeting sequence, a linker, an epitope tag, a detectable fusion partner, such as a fluorescent protein, β-galactosidase, luciferase and/or functionally similar peptides. Components of a fusion protein may be joined by one more peptide bonds, disulfide linkages, and/or other covalent bonds.

In the context of the present disclosure, “immobilized” refers to covalent attachment of an enzyme to a solid support with or without a linker. Examples of solid supports include beads (e.g., magnetic, agarose, polystyrene, polyacrylamide, chitin). Beads may include one or more surface modifications (e.g., O⁶-benzyleguanine, polyethylene glycol) that facilitate covalent attachment and/or activity of an enzyme of interest. For example, a support may comprise a ligand and an enzyme may have a receptor for such ligand or an enzyme may comprise a ligand and a support may comprise a receptor for such ligand. Receptor-ligand binding may be covalent or non-covalent. Non-covalent attachment (e.g., avidin:biotin, chitin:CBP) may be useful in some embodiments, for example, where the level of dissociation of the binding partner is deemed tolerable. A linker may be disposed between a support and an enzyme. For example, linker disposed between a support and an enzyme may have a first covalent bond to the support and a second covalent bond to the enzyme. An immobilized enzyme comprising a ligand-receptor attachment may have a linker disposed between the support and the ligand-receptor attachment, a linker disposed between the enzyme and the ligand-receptor attachment, or both. An immobilized enzyme comprising a linker may also comprise an optional covalent bond directly between the enzyme and the support. A linker may be of any desired length and have any desired range of motion. A peptide linker may comprise one or more repeats (e.g., 1-10 repeats) of glycine-serine.

In the context of the present disclosure, “modified nucleotide” refers to nucleotides having a modification on the sugars (e.g., 2′-fluororibose, ribose, 2′-deoxyribose, arabinose, and hexose); and/or in the phosphate groups (e.g., phosphorothioates and 5′-N-phosphoramidite linkages); and/or in the nucleotide base (e.g., as described in U.S. Pat. No. 8,383,340; WO 2013/151666; U.S. Pat. No. 9,428,535 B2; US 2016/0032316). Examples of modified nucleotides include pseudouridine and N1-methyl-pseudouridine.

In the context of the present disclosure, “non-naturally occurring” refers to a molecule (e.g., a polynucleotide, polypeptide, carbohydrate, or lipid) or composition that does not exist in nature. Such a molecule or composition may differ from naturally occurring molecules or compositions in one or more respects. For example, a polymer (e.g., a polynucleotide, polypeptide, or carbohydrate) may differ in the kind and arrangement of the component parts (e.g., nucleotide sequence, amino acid sequence, or sugar molecules). A polymer may differ from a naturally occurring polymer with respect to the molecule(s) to which it is linked. For example, a “non-naturally occurring” polypeptide (e.g., protein) may differ from naturally occurring polypeptides in its secondary, tertiary, or quaternary structure, by having (or lacking) a chemical bond (e.g., a covalent bond including a peptide bond, a phosphate bond, a disulfide bond, an ester bond, and ether bond, and others) to a lipid, a carbohydrate, a second polypeptide (e.g., a fusion protein), or any other molecule. Similarly, a “non-naturally occurring” polynucleotide or nucleic acid may comprise (or lack) one or more other modifications (e.g., an added label or other moiety) to the 5′-end, the 3′ end, and/or between the 5′- and 3′-ends (e.g., methylation) of the nucleic acid. A “non-naturally occurring” molecule or composition may differ from naturally occurring compositions in one or more of the following respects: (a) having components that are not combined in nature, (b) having components in ratios and/or concentrations not found in nature, (c) lacking one or more components otherwise found in naturally occurring molecules or compositions (e.g., a cell-free composition, a chromosome-free composition, a histone-free composition, a polymerase-free composition, a cell membrane-free composition), (d) having a form not found in nature (e.g., dried, freeze dried, lyophilized, crystalline, aqueous, immobilized), and (e) having one or more additional components beyond those found in nature (e.g., a buffering agent, a detergent, a dye, a solvent or a preservative).

With reference to an amino acid, “position” refers to the place such amino acid occupies in the primary sequence of a peptide or polypeptide numbered from its amino terminus to its carboxy terminus. A position in one primary sequence may correspond to a position in a second primary sequence, for example, where the two positions are opposite one another when the two primary sequences are aligned using an alignment algorithm (e.g., BLAST (Journal of Molecular Biology. 215 (3): 403-410) using default parameters (e.g., expect threshold 0.05, word size 3, max matches in a query range 0, matrix BLOSUM62, Gap existence 11 extension 1, and conditional compositional score matrix adjustment) or custom parameters). An amino acid position in one sequence may correspond to a position within a functionally equivalent motif or structural motif that can be identified within one or more other sequence(s) in a database by alignment of the motifs.

As used herein, “salt” refers to a material comprising an organic or inorganic cation of a base (e.g., Na⁺, Ca²⁺, K⁺, Mn⁺, Mg²⁺) and an organic or inorganic anion of an acid (e.g., Cl⁻, CO₃²⁻, NO₃⁻, SO₄²⁻, CH₃COO⁻). Examples of salt include NaCl, KCl, CaCl₂, MgCl₂, Mg(NO₃)₂, MnSO₄, K₂SO₄, and NaHCO₃. In the context of salt tolerance and salt indifference, a salt may be a monovalent salt (e.g., NaCl), a divalent salt (e.g., MgCl₂, CaCl₂), an organic salt (NaCH₃COO), and/or an inorganic salt.

As used herein, “salt indifferent” refers to a property or capacity to display activity both in the absence of salt and across a range of concentrations of one or more salts. A salt indifferent salt active nuclease variant, for example, may display DNA-binding activity and/or catalytic activity in the absence or presence of one or more salts (e.g., from 0 M to 1 M salt) that is at least 20%, at least 25%, at least 30%, at least 35%, at least 40%, at least 45%, at least 50%, at least 55%, or at least 60% of such variant's peak activity. For example, a salt active nuclease variant may have at least 20% of its peak catalytic activity under conditions spanning a range of total salt from 0 M to 1 M. Salt indifferent enzymes may be distinguished from salt sensitive enzymes, which have little or no activity (e.g., less than 20% of peak catalytic activity) in the absence of salt (salt-requiring enzymes) or have little or no activity (e.g., less than 20% of peak catalytic activity) in the presence of salt (salt-labile enzymes).

As used herein, “salt tolerant” refers to a property or capacity to display activity in the presence of one or more salts. A salt tolerant salt active nuclease variant, for example, may display DNA-binding activity and/or catalytic activity in the presence of one or more salts. Binding activity may be evaluated in suitable biochemical terms, for example, binding affinity (Kd). Similarly, catalytic activity may be evaluated in suitable biochemical terms, for example, Michaelis constant (Km) and/or maximal reaction velocity (Vmax). Catalytic activity and/or DNA binding of a salt tolerant salt active nuclease variant may be less sensitive to the presence of salt than a reference enzyme (e.g., a corresponding wild-type enzyme). A salt tolerant salt active nuclease variant, for example, may bind a (single stranded or double stranded) nucleic acid (DNA and/or RNA) and/or hydrolyze the nucleic acid to yield products comprising at least one 5′-phosphorylated nucleic acid strand and/or at least one 3′-hydroxylated nucleic acid strand in the presence of at least 150 mM, at least 250 mM, at least 500 mM, or at least 900 mM salt. Catalytic activity of a salt tolerant salt active nuclease variant in the presence of 200 mM salt may be at least 50%, at least 60%, at least 70%, at least 80%, at least 90%, at least 95%, or at least 98% of the activity of the same salt tolerant salt active nuclease variant in the absence of salt when assayed by any method disclosed herein. A salt tolerant salt active nuclease variant may display at least 20%, at least 22%, at least 24%, at least 26%, at least 28%, at least 30%, at least 35%, at least 40%, or at least 50% of its peak catalytic activity in the presence of up to 1 M salt.

A salt active nuclease variant (e.g., in the presence of 100 mM, 200 mM, 300 mM, 400 mM, 500 mM salt) may be less processive than a corresponding reference (e.g., wild-type) enzyme under the same conditions with or without a measurable difference in overall catalytic activity. For example, a salt active nuclease variant may bind and release substrate molecules more frequently than a corresponding reference (e.g., wild-type) enzyme under the same conditions. Short substrates may obscure this property with both variant and reference enzymes appearing to cut the substrate equally well. One approach to resolving this difference includes contacting each enzyme with a mixture of two or more substrates, wherein a first substrate is short (e.g., ≤100 nts) and comprises a detection system and a second substrate is long (e.g., ≥500 nts) without a detection system. The long substrate may be included in molar excess over the short substrate. Without limiting any embodiment to any particular mechanism of action, a less processive endoribonuclease is expected to switch substrates more frequently and digest (e.g., at least once) more substrate molecules in the mixture compared to a more processive enzyme.

In the context of the present disclosure, “salt active nuclease” refers to any enzyme that digests one or more salt active nuclease substrates (e.g., single- and double-stranded DNA and RNA, including, in each case, linear and circular forms) in the presence of salt (e.g., total salt ≥50 mM, ≥100 mM, ≥150 mM, ≥200 mM, ≥250 mM, ≥300 mM, ≥350 mM, ≥400 mM, ≥450 mM, ≥500 mM, ≥600 mM, ≥700 mM, ≥800 mM, ≥900 mM, or ≥1 M salt) and optionally in the absence of salt.

In the context of the present disclosure, “salt active nuclease substrate” refers to a molecule having at least one bond that is cleavable by a salt active nuclease and/or a salt active nuclease variant. Examples of salt active nuclease substrates include single-stranded DNA, double-stranded DNA, single-stranded RNA, double-stranded RNA, and RNA:DNA duplexes. Examples of salt active nuclease substrates include linear, circular, and branched polynucleotides. Salt active nuclease substrates may be associated (e.g., non-covalently bound) with one or more other materials (e.g., biological materials such as proteins, lipids, and/or carbohydrates, non-biological materials such as magnetic beads or other supports).

In the context of the present disclosure, “salt active nuclease variant” refers to any non-naturally occurring salt active nuclease that digests one or more salt active nuclease substrates (e.g., single- and double-stranded DNA and RNA) in the presence of salt (e.g., total salt ≥150 mM, ≥200 mM, ≥250 mM, ≥300 mM, ≥350 mM, ≥400 mM, ≥450 mM, ≥500 mM, ≥600 mM, ≥700 mM, ≥800 mM, ≥900 mM, or ≥1 M salt) and optionally in the absence of salt. A salt active nuclease variant, in some embodiments, may have an amino acid sequence sharing any desired degree of sequence identity with positions 22-234 of SEQ ID NO:5 up to (but excluding) 100% identity. According to some embodiments, a salt active nuclease variant may comprise an amino acid sequence having ≥85%, ≥88%, ≥90%, ≥92%, ≥93%, ≥95%, ≥96%, ≥97%, ≥98% ≥99% identity to one or more of SEQ ID NOS:1-5 and have catalytic activity (e.g., ssDNA nuclease activity, dsDNA nuclease activity, ssRNA nuclease activity, ssRNA nuclease activity, and/or duplex DNA:RNA nuclease activity). For example, salt active nuclease variant may comprise an amino acid sequence having a serine (e.g., a Q->S substitution or another conservative substitution) at a position corresponding to position 52 of SEQ ID NO:1 (a “Q52S” substitution) and/or corresponding to position 73 of SEQ ID NO:5 (a “Q73S” substitution). In some embodiments, a salt active nuclease variant may comprise an amino acid sequence having at least 96%, at least 97%, at least 98% or at least 99% identity to one or more of SEQ ID NOS:1-4, wherein the amino acid at the position corresponding to position 52 of any of SEQ ID NOS:1-4 is not Q (e.g., is instead C, D, E, H, K, N, P, R, S, T or Y). In some embodiments, a salt active nuclease variant may comprise an amino acid sequence having at least 96%, at least 97%, at least 98% or at least 99% identity to SEQ ID NO:3, wherein the amino acid at the position corresponding to position 53 of any of SEQ ID NO:3 is not Q (e.g., is instead C, D, E, H, K, N, P, R, S, T or Y). In some embodiments, a salt active nuclease variant may comprise an amino acid sequence having ≥85%, ≥88%, ≥90%, ≥92%, ≥93%, ≥95%, ≥96%, ≥97%, ≥98% ≥99% identity to positions 22-234 of SEQ ID NO:4, wherein the amino acid at the position corresponding to position 73 of SEQ ID NO:4 is not Q (e.g., is instead C, D, E, H, K, N, P, R, S, T or Y). A salt tolerant nuclease variant, according to some embodiments, may comprise an amino acid sequence having ≥85%, ≥88%, ≥90%, ≥92%, ≥93%, ≥95%, ≥96%, ≥97%, ≥98% ≥99% identity to any of SEQ ID NOS:1-4, wherein each substitution (e.g., relative to amino acids 22-234 of SEQ ID NO:5) is a conservative substitution or wherein all substitutions are conservative substitutions except one (which is non-conservative) or wherein all substitutions are conservative substitutions except two or wherein all substitutions are conservative substitutions except three or wherein all substitutions are conservative substitutions except four or wherein all substitutions are conservative substitutions except five.

A salt tolerant nuclease variant may be salt tolerant and/or salt indifferent. Catalytic activity of a salt active nuclease variant may persist across a range of salt concentrations, temperatures and/or pH. For example, a salt active nuclease variant may display catalytic activity under such a range of conditions and/or following removal from exposure to conditions within such a range. A salt active nuclease variant may have catalytic activity at and/or following exposure to temperatures in ranges X to Y, where X is any of 1° C., 2° C., 4° C., 10° C., 15° C., 20° C., 25° C., 30° C. and Y is any of 25° C., 30° C., 35° C., 40° C., 45° C., 50° C., 55° C., 60° C., 65° C. and X<Y. For example, a salt active nuclease variant may have catalytic activity at and/or following exposure to temperatures in ranges 1° C.-65° C., 2° C.-60° C., 10° C.-60° C., 20° C.-55° C., 35° C.-55° C. According to some embodiments, a salt active nuclease variant has higher (e.g., ≥25%, ≥35%, ≥50%, ≥75% higher) activity at 250 mM salt and 50° C. than a corresponding wild type enzyme. A salt active nuclease variant may have catalytic activity at and/or following exposure to a pH in ranges X^{†} to Y^{†}, where X^{†} is any of 6.5, 7, 7.5, 8, 8.5 and Y^{†} is any of 8, 8.5, 9, 9.5, 10, 10.5, 11, 11.5, 12 and X^{†}<Y^{†}. For example, a salt active nuclease variant may have catalytic activity at and/or following exposure to pH in ranges 6.5-12, 7-11.5, 7.5-11, 7.5-10.5, 8-10, 8.5-9.5.

A salt active nuclease variant may comprise one or more amino acids in addition to a SEQ ID NOS:1-5. For example, a salt active nuclease variant may comprise (e.g., at its amino terminal end or carboxy terminal end) 1-25 amino acids. Such additional amino acids may enable, facilitate and/or enhance translation, expression, cellular sorting, inactivation (e.g., by including a protease recognition and/or cleavage site), and/or purification. Such additional amino acids may constitute a linker, for example, to a support (e.g., a magnetic bead) or another protein.

A salt active nuclease variant may have one or more other desirable properties (beyond retaining activity in high salt compositions) including, for example, readily binding cation exchange media attributable to its high pI (e.g., 9≤pI≤10), reduced binding affinity to actin, mucolytic activity, and/or phosphodiesterase/hydrolytic activity.

In the context of the present disclosure, “substitution” refers to an amino acid residue at a position in a comparator amino acid sequence that differs with respect to a corresponding position of a reference amino acid sequence, where the comparator and reference sequences are at least 60% identical to each other or at least 70% identical to each other or at least 80% identical to each other. A reference sequence and comparator sequence may have the same length or similar lengths (e.g., differing by ≤12%, ≤5%, ≤1%). A substitute amino acid residue at a position, in addition to differing from the corresponding position of a reference amino acid sequence, may differ from the amino acid at the corresponding position of all naturally-occurring sequences that are at least 60% identical to each other or at least 70% identical to each other or at least 80% identical to the reference sequence. Optionally, a substitute amino acid may have different properties than the amino acid in the corresponding position of the reference sequence. Optionally, a substitute amino acid may have similar properties to the amino acid in the corresponding position of the reference sequence (a “conservative” substitution). For example, a non-polar amino acid (e.g., A, V, L, I, M, W, and F (and optionally C, G, and P) may substitute for another non-polar amino acid; a polar amino acid (e.g., N, Q, S, T, and Y) may substitute for another polar amino acid (e.g., C, D, E, H, K, N, P, Q, R, S, T, and Y); a positively charged amino acid (e.g., H, K, and R) may substitute for another positively charged amino acid; and a negatively charged amino acid (e.g., D and E) may substitute for another negatively charged amino acid. A substitute amino acid may be a natural amino acid (e.g., replacing another natural amino acid or a non-natural amino acid). A substitute amino acid may be a non-natural amino acid (e.g., replacing a natural amino acid or another non-natural amino acid). A comparator sequence may differ from a reference sequence, at a corresponding position, by having a substitution, an insertion, or a deflection.

All publications, patents, and patent applications mentioned in this specification are incorporated herein in their entirety by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

### Compositions

The present disclosure relates, in some embodiments, to compositions for reducing the amount (e.g., molar amount) of a polynucleotide in a sample and/or samples having one or more polynucleotides. Compositions may comprise, according to some embodiments, any of the salt active nuclease variants disclosed herein, a salt active nuclease substrate (e.g., ssRNA, dsRNA, ssDNA, and/or dsDNA), and, optionally, one or more salts. Salt active nuclease variant compositions may comprise, in some embodiments, any of the salt active nuclease variants disclosed herein (e.g., variants having an amino acid sequence sharing any desired degree of sequence identity with positions 22-234 of SEQ ID NO:5 up to (but excluding) 100% identity), a salt active nuclease substrate, and one or more of a salt (e.g., NaCl, MgCl₂), a protein (e.g., albumin, topoisomerase, polymerase), DNA, RNA, a buffering agent, a cell (e.g., intact or digested), a biological fluid or secretion (e.g., mucus, pus), and/or (non-naturally occurring) combinations thereof. Compositions may comprise one or more salts (e.g., any of the salts disclosed here alone or in any combination) at a total salt concentration of, for example, ≥150 mM, ≥200 mM, ≥250 mM, ≥300 mM, ≥350 mM, ≥400 mM, ≥450 mM, ≥500 mM, ≥600 mM, ≥700 mM, ≥800 mM, ≥900 mM, or ≥1 M. Salt active nuclease variants included in compositions may comprise an amino acid sequence having ≥85%, ≥88%, ≥90%, ≥92%, ≥93%, ≥95%, ≥96%, ≥97%, ≥98% ≥99% identity to one or more of SEQ ID NOS:1-5 and have catalytic activity (e.g., ssDNA, dsDNA, ssRNA, ssRNA, and/or duplex DNA:RNA nuclease activity). In each case, salt present may be a single salt species or a mixture of salts. In each case, salt present may comprise monovalent and/or divalent salts. A salt active nuclease variant composition may comprise one or more ionic, non-ionic, and/or zwitterionic detergents (e.g., octoxinol, polysorbate 20), crowding agents, sugars, starches, cellulose, lipids, and oils.

Salt active nuclease variants and compositions thereof may have any desirable degree of purity including, for example, cell paste, crude extract, partially purified, and/or purified preparations. Salt active nuclease variants and compositions thereof may have any desirable form including, for example, a liquid, a gel, a film, a powder, a cake, and/or any dried or lyophilized form. A salt active nuclease variant composition may comprise one or more stabilizers including, for example, an aptamer, a monosaccharide, a disaccharide, a trisaccharide, a tetrasaccharide, starch, cellulose, dextrin, and dextran.

In some embodiments, a salt active nuclease variant may be encoded by a nucleic acid sequence having ≥85%, ≥88%, ≥90%, ≥92%, ≥93%, ≥95%, ≥96%, ≥97%, ≥98% ≥99% identity to a sequence (e.g., a codon optimized sequence) encoding the amino acid sequence of any of SEQ ID NOS:1-5. For example, a salt active nuclease variant may be encoded by a nucleic acid sequence having ≥85%, ≥88%, ≥90%, ≥92%, ≥93%, ≥95%, ≥96%, ≥97%, ≥98% ≥99% identity to SEQ ID NO:10. A nucleic acid encoding a salt active nuclease variant may be included in an expression cassette, expression vector, or other expressible form suitable for in vitro or in vivo expression (e.g., in E. coli or other bacteria or P. pastoris or other yeast).

### Kits

The present disclosure further relates to kits including salt active nuclease variants. For example, a kit may include a salt active nuclease variant, other enzymes (e.g., polymerases, enzymes other than polymerases, or both), buffering agents, or combinations thereof. Enzymes may be included in a storage buffer. Any suitable storage buffer may be used, for example, buffers comprising one or more of a cryoprotectant (e.g., a polyol such as glycerol, an antifreeze protein), a salt, a detergent, a reducing agent, a sugar, a chelator, and an antimicrobial agent and having a pH tolerated by the enzyme to be stored, for example, between pH 6 and 9. A composition or kit may include a reaction buffer which may be in concentrated form, and the buffer may contain additives (e.g. glycerol), salt (e.g. NaCl, KCl), reducing agent, EDTA or detergents, among others. Detergents include nonionic detergents (e.g., t-octylphenoxypolyethoxyethanol), anionic detergents (e.g., alkylbenzene sulfonates), cationic detergents (e.g., alkylbenzene quaternary ammonium), and zwitterionic detergents. A composition or kit comprising dNTPs may include one, two, three of all four of dATP, dTTP, dGTP and dCTP. A kit comprising rNTPs may include one, two, three of all four of rATP, rUTP, rGTP and rCTP. A kit may further comprise one or more modified nucleotides. A kit may optionally comprise one or more primers (random primers, bump primers, exonuclease-resistant primers, chemically-modified primers, custom sequence primers, or combinations thereof).

A kit may be a non-natural collection of components configured, for example, for convenient storage, shipping, delivery, and/or use. One or more components of a kit may be included in one container for a single step reaction, or one or more components may be contained in one container, but separated from other components for sequential use or parallel use. The contents of a kit may be formulated for use in a desired method or process.

A kit is provided that contains: (i) a salt active nuclease variant; and (ii) a buffer. The salt active nuclease variant may have a lyophilized form or may be included in a buffer (e.g., a storage buffer or a reaction buffer in concentrated form). A kit may contain the salt active nuclease variant in a mastermix suitable for receiving and digesting a nucleic acid. A salt active nuclease variant may be a purified enzyme so as to contain substantially no DNA or RNA and no nucleases. The reaction buffer in (ii) and/or storage buffers containing the DNA polymerase in (i) may include non-ionic, ionic e.g. anionic or zwitterionic surfactants and crowding agents. A kit may include the salt active nuclease variant and the reaction buffer in a single tube or in different tubes.

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or subpackaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### Methods

Salt active nuclease variants disclosed herein may be useful in many molecular, cellular, and therapeutic applications, processes, methods, and/or workflows. For example, salt active nuclease variants may be use in methods and/or workflows that include, for example, strand displacement, nick translation, in vitro transcription, in vitro protein expression (e.g., cell free protein expression, PURExpress®) DNA fragmentation, footprinting, PCR (e.g., RT-PCR), RNA sequencing, and RNA purification. Salt active nuclease variants may be used, in some embodiments, to digest DNA (e.g., dsDNA and/or ssDNA) and/or RNA (e.g., dsRNA and/or ssRNA) where its presence may impair or complicate analysis (e.g., of other components of a sample). The present disclosure provides, in some embodiments, methods comprising contacting a salt active nuclease variant with a molecule comprising a polydeoxyribonucleotide. For example, a method may comprise contacting a salt active nuclease variant with a composition comprising DNA and at least one non-DNA species to digest the DNA present. Such action on the DNA present may leave the non-DNA species unmodified or substantially unmodified (e.g., as to structure, composition, and/or concentration).

The present disclosure relates, in some embodiments, to methods comprising contacting a salt active nuclease variant with a composition comprising a polynucleotide and at least one material of interest to form polynucleotide cleavage products without modifying the material of interest. For example, a method may comprise contacting a salt active nuclease variant with a composition comprising DNA and/or RNA molecules and a protein (or vitamins or saccharides or polysaccharides or lipids or cellular metabolites or any other molecule of interest other than polynucleotides) to digest the DNA and/or RNA molecules present without modifying the protein (or another non-polynucleotide molecule of interest). Such contacting may be included in methods for cleaning up protein after isolation from a cell or tissue, after in vitro transcription, prior to elution from a solid support, prior to amino acid sequencing, for preparing protein samples for separation on 2-D gels, and/or for identifying protein binding sequences on DNA (salt active nuclease footprinting). In some embodiments, the salt active nuclease variant may be included in any desirable degree of purity (e.g., as cell paste, crude extract, partially purified, and/or purified preparations). The protein of interest may remain unaltered or substantially unaltered. For example, contacting a salt active nuclease variant with a composition comprising a protein (or another non-polynucleotide molecule of interest) and DNA and/or RNA may result in a product composition comprising over 50%, over 80%, over 85%, over 90%, over 95%, or over 99% of the starting, intact protein (or another non-polynucleotide molecule of interest) and/or less than 50%, less than 25%, less than 10%, less than 5%, less than 1%, less than 0.5%, or less than 0.1% of the starting, intact DNA and less than 50%, less than 25%, less than 10%, less than 5%, less than 1%, less than 0.5%, or less than 0.1% of the starting, intact RNA. A method may include fracturing one or more cells to form the composition comprising the protein (or another non-polynucleotide molecule of interest) and DNA and/or RNA, wherein at least a portion of the protein (or another non-DNA molecule of interest) comprises cellular protein (or another cellular, non-DNA molecule of interest), at least a portion of the DNA comprises cellular DNA, and at least a portion of the RNA comprises cellular RNA. The size of digestion products and/or degree of digestion may be managed, for example, by selecting the salt active nuclease variant with a desired activity, increasing or decreasing the concentration of the selected salt active nuclease variant, increasing or decreasing the incubation time or temperature, increasing or decreasing magnesium concentration, increasing or decreasing salt concentration, and/or increasing or decreasing the pH. In some embodiments, methods may be adapted to digest DNA as fully as practicable or digest DNA (non-specifically) into fragments within a selected range of sizes.

A salt active nuclease variant may be useful in digesting and/or removing unwanted polynucleotides from a composition comprising liposomes, micelles, phage particles, and/or viral particles. For example, a desirable polynucleotide (e.g., a therapeutic RNA or DNA) may be packaged in a micelle, liposome, phage particle, or viral particle using a protocol which does or may result in a packaged composition comprising some packaged polynucleotide and some residual unpackaged polynucleotide (or fragments thereof). According to some embodiments, a method may comprise contacting a salt active nuclease variant with such packaged composition (e.g., comprising unpackaged polynucleotides) to produce a product composition, wherein the product composition comprises less unpackaged polynucleotide than the packaged composition.

In some embodiments, a salt active nuclease variant may be contacted with a composition comprising DNA (e.g., genomic fragments or other long (≥10 kb) DNA fragments) to digest such DNA. Such contacting may be included in methods for creating a fragmented DNA library and methods of cell culture preparation (e.g., tissue disaggregation), cultivation, manipulation, and storage to reduce or prevent cell clumping.

A salt active nuclease variant may be used in connection with in vitro transcription (IVT), according to some embodiments. IVT methods may include contacting a DNA template (e.g., a double stranded DNA comprising a coding sequence and an expression control sequence operably linked to the coding sequence) with an RNA polymerase (e.g., T7 RNA polymerase) optionally in the presence of NTPs, salt, and/or a reaction buffer to form a transcription product composition comprising a transcription product (e.g., RNA) and the DNA template. The resulting RNA may be translated into protein by any available method, for example, PURExpress® (New England Biolabs, Inc.). IVT methods, in some embodiments, may comprise contacting a salt active nuclease variant with the DNA template to digest the DNA template and form a digested composition comprising DNA template digestion products and/or RNA digestion products. IVT methods may comprise separating the protein product from one or more of the other components of the transcription/translation product composition or the digested composition, for example, the DNA template, DNA template digestion products, the RNA polymerase, NTPs, salt, and reaction buffer. Techniques for such separation include column purification, phase separation (e.g., phenol-chloroform), and fractionation (e.g., size, charge, hydrophobicity, polarity, and ratios thereof) among others.

According to some embodiments, contacting a DNA and/or RNA with a salt active nuclease variant may (further) comprise contacting the polydeoxyribonucleic acid with the salt active nuclease variant in the presence of total salt ≥50 mM, ≥100 mM, ≥150 mM, ≥200 mM, ≥250 mM, ≥300 mM, ≥350 mM, ≥400 mM, ≥450 mM, ≥500 mM, ≥600 mM, ≥700 mM, ≥800 mM, ≥900 mM, or ≥1 M salt. In each case, salt present may be a single salt species or a mixture of salts. In each case, salt present may comprise monovalent and/or divalent salts.

The present disclosure further relates to methods of making a salt active nuclease variant. For example, a salt active nuclease variant may be produced by in vitro transcription and/or in vitro translation (e.g., PURExpress®, New England Biolabs, Inc.). In some embodiments, a salt active nuclease variant may be produced in vivo. For example, a method may include (a) culturing a host cell comprising an expression vector or expression cassette comprising a nucleic acid sequence having at least 80%, at least 85%, at least 90%, at least 93%, at least 96%, at least 97%, at least 98% or at least 99% identity to a sequence (e.g., a codon optimized sequence) encoding the amino acid sequence of SEQ ID NO:1, 2, 3, and/or 4 operably linked to an expression control sequence to produce a cultured host cell composition comprising the salt active nuclease variant, and (optionally) isolating the salt active nuclease variant from the cultured host cell composition (e.g., culture supernatant, culture lysate, culture cell paste).

### EXAMPLES

Some specific example embodiments may be illustrated by one or more of the examples provided herein.

### Example 1: Nuclease Activity on dsDNA, ssDNA, and RNA Substrates in NaCl

Example endonucleases (e.g., vsEndA and vsEndA-Q52S) were tested with substrates and under conditions disclosed in this and subsequent examples and the resulting performance compared. vsEndA and vsEndA-Q52S were expressed in E. coli. For this, the 2 codon optimized genes vsendA and vsEndA-Q52S were cloned in a high copy plasmid under control of an inducible promoter.

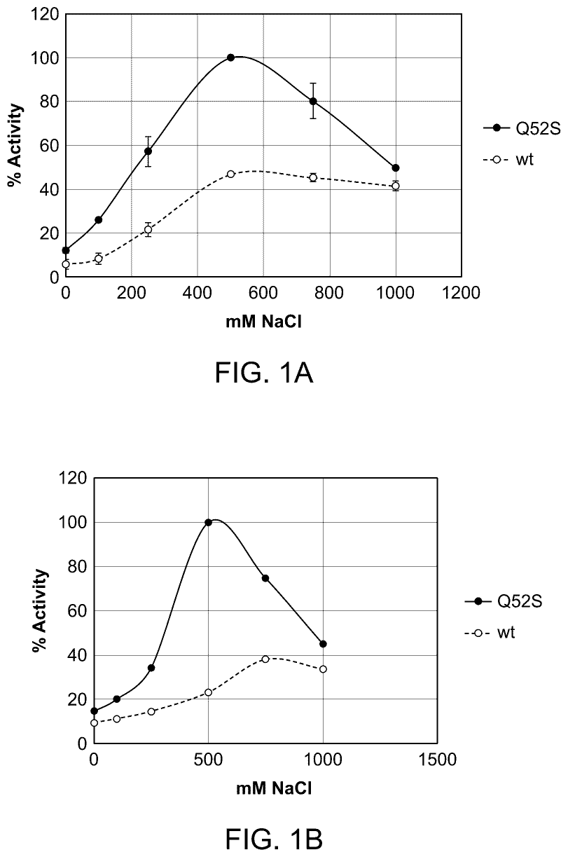

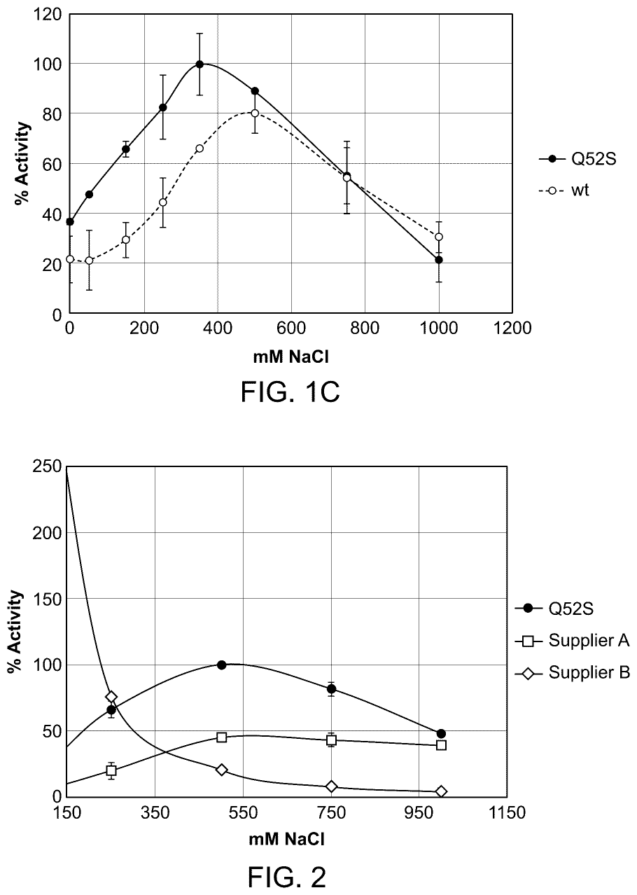

For testing vsEndA and vsEndA-Q52S activity on dsDNA, ssDNA and RNA fluorescent substrates, both enzymes were diluted to a working concentration of 0.1 μg/mL in 25 mM Tris-HCl pH 8.5, 5 mM MgCl₂ supplemented with NaCl at 0, 100, 250, 500, 750 and 1000 mM NaCl and mixed with 5 μM of a dsDNA substate (a 35mer FAM-BHQ1 labeled hairpin oligonucleotide (SEQ ID NO:7)) or 5 μM of a ssDNA substrate (a linear 15mer oligo substrate (SEQ ID NO:8)) or 0.2 μM of RNA fluorescent substrate, and with 1 mg/mL of calf-thymus DNA. Each 50 μL reaction was loaded into a well of a 96 well plate half area black bottom and incubated at 25° C. on a SpectraMax (Molecular Devices) to follow the kinetic at Ex 485 nm-Em 525 nm cutoff 515 nm every 30 sec for 30 min. As measurements were performed 2 times, the released fluorescence values chosen at a specific endpoint in the linear range were averaged and converted to percentages of activity using the highest value as 100%. Results are shown in FIG. 1.

On DNA substrates (dsDNA and ssDNA), the activities of both enzymes peak at 500 mM NaCl under the conditions tested. When tested on dsDNA (FIG. 1A), vEndA-Q52S exhibits 4× increased activity at 100 mM NaCl, 2× at 500 mM and 1.6×750 mM NaCl compared to vsEndA. On a ssDNA substrate (FIG. 1B), vEndA-Q52S shows 2× improvement compared to vsEndA at 100 mM, 4× at 500 mM and 750 mM NaCl. Finally, on the RNA substrate (FIG. 1C), vEndA-Q52S is ~2× more performant than vsEndA in NaCl <350 mM. Overall, these data show that, when tested on fluorescent substrates (dsDNA, ssDNA and RNA) in presence of calf-thymus DNA, vsEndA-Q52S outperforms vsEndA wild-type by 2-4× between 250 and 750 mM NaCl in a Tris buffer pH 8.5.

### Example 2: Nuclease Activity of a SAN Variant and 2 Other Endonucleases in NaCl

Activities of an example salt active nuclease variant and 2 commercially available endonucleases were compared in the presence of NaCl. vsEndA-Q52S, an endonuclease from Supplier A and an endonuclease from Supplier B were each diluted to a working concentration of 0.1 μg/mL in their respective buffer (vsEndA-Q52S and Supplier A: 25 mM Tris-HCl pH 8.5, 5 mM MgCl₂, Supplier B: 50 mM Tris-HCl pH 8, 1 mM MgCl₂) supplemented with 100, 250, 500, 750 and 1000 mM NaCl, and mixed with 5 μM of the dsDNA substrate of Example 1 and 0.5 mg/mL of calf-thymus DNA, in 50 μL final. Each reaction was loaded on a 96 well plate half area black bottom and incubated at 25° C. in a SpectraMax (Molecular Devices) to follow the kinetics as described in Example 1.

The endonuclease from Supplier B shows decreased activity as the concentration of NaCl is increased. On the other hand, both vsEndA-Q52S and Supplier A activity increases as the NaCl concentration increases, to a peak at 500 mM NaCl followed by a slight decrease (not less than 50%) between 500 mM and 1000 mM NaCl. vsEndA-Q52S is consistently 2× more active than the Supplier A endonuclease (FIG. 2). Overall, vsEndA-Q52S outperforms Supplier A and Supplier B endonucleases by 2× to 4× between 300 mM and 1000 mM NaCl.

### Example 3: pH Spectrum of Nuclease Activity on dsDNA, ssDNA, and RNA Substrates

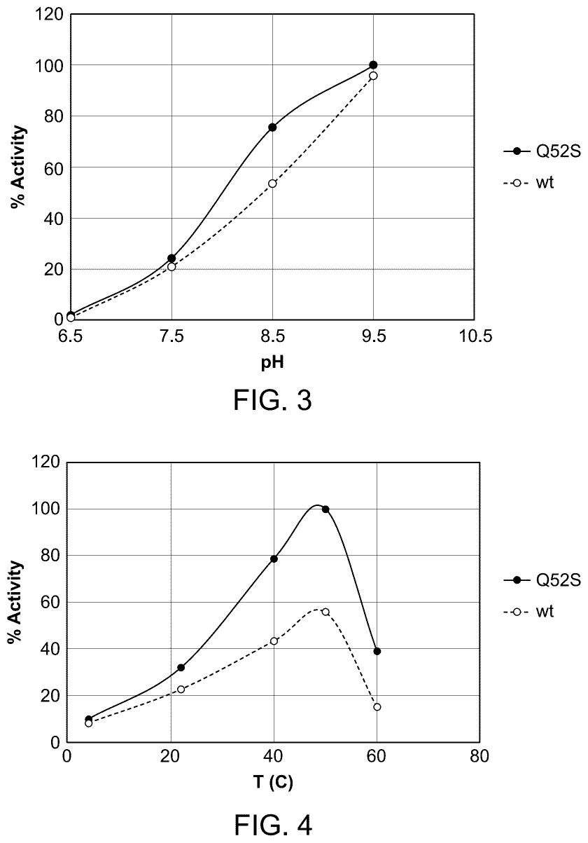

Nuclease activity was assessed in 25 mM Tris-HCl, 500 mM NaCl, 5 mM MgCl₂ buffer across a pH range of 6.5 to 9.5. Enzymes vsEndA and vsEndA-Q52S were diluted to a working concentration of 0.1 μg/mL in each buffer and mixed with 5 μM of the dsDNA substrate of Example 1 or 5 μM of the linear ssDNA substrate of Example 1 or 0.2 μM of RNA fluorescent substrate, the RNA oligonucleotide substrate v2 from the Kit RNaseAlert™ QC System v2 (Thermofisher), along with 1 mg/mL of calf-thymus DNA. The 50 μL reactions were loaded on a 96 well plate half area black bottom and incubated at 25° C. in a SpectraMax (Molecular Devices) to follow the kinetics as described in Example 1. Results are shown in FIG. 3 and TABLE 1.

When tested in Tris buffer pH 6.5 to 9.5 with 500 mM NaCl and 5 mM MgCl₂ on a dsDNA substrate and calf-thymus DNA, vsEndA-Q52S showed similar activity to vsEndA, except at pH 8.5, where vsEndA-Q52S is 40% more active than vsEndA (FIG. 3).

TABLE 1
Evaluation of vsEndA and vsEndA-Q52S activity
at diverse pHs on various substrates.

| pH | dsDNA | ssDNA | RNA |
|---|---|---|---|
| 6.5 | − | − | − |
| 7 | + | + | − |
| 7.5 | ++ | ++ | − |
| 8 | +++ | +++ | + |
| 8.5 | +++ | +++ | +++ |
| 9 | ++ | +++ | + |
| 9.5 | ++ | ++ | − |

The respective activity of each enzyme was evaluated independently as follows +++ >80% activity, ++ >50% activity, + >10% activity, − <10% activity measured.

vsEndA and vsEndA-Q52S activities are both optimal at pH 8.5 on all 3 substrates. On dsDNA and ssDNA, the activity is over 5% between pH 7 and 9.5, while on RNA, the peak is narrower between 8 and 9 in the conditions tested (TABLE 1).

### Example 4: Thermal Spectrum of Nuclease Activity on dsDNA, ssDNA, and RNA

A temperature screen was carried out by mixing vsEndA or vsEndA-Q52S with DNA substrates in 25 mM Tris-HCl pH 8.5, 500 mM NaCl, 5 mM MgCl₂ followed by an incubation at 4° C., 20° C., 40° C., 50° C. or 60° C. for 10 min. Both enzymes were diluted at 0.1 μg/mL in 25 mM Tris-HCl pH 8.5, 500 mM NaCl, 5 mM MgCl₂ and mixed with 5 μM of the dsDNA substrate of Example 1 and 1 mg/mL of calf-thymus DNA. The 50 μL reactions were set up in PCR strip tubes and incubated at 4° C., 20° C., 40° C., 50° C. and 60° C. in a PCR instrument (Bio-Rad) for 10 min. The samples were immediately transferred on a 96 well plate half area black bottom to measure the fluorescence released on a SpectraMax (Molecular Devices) as described in Example 1. The values of released fluorescence were converted to percentages of activity using the highest value as 100% (FIG. 4).

The activity of both enzymes vsEndA and vsEndA-Q52S peaks at 50° C. when tested on DNA in a Tris buffer pH 8.5 with 500 mM NaCl. vsEndA-Q52S systematically outperforms vsEndA by 1.2 to 2× at all temperature tested (FIG. 4).

### Example 5: Nuclease Activity on λ DNA

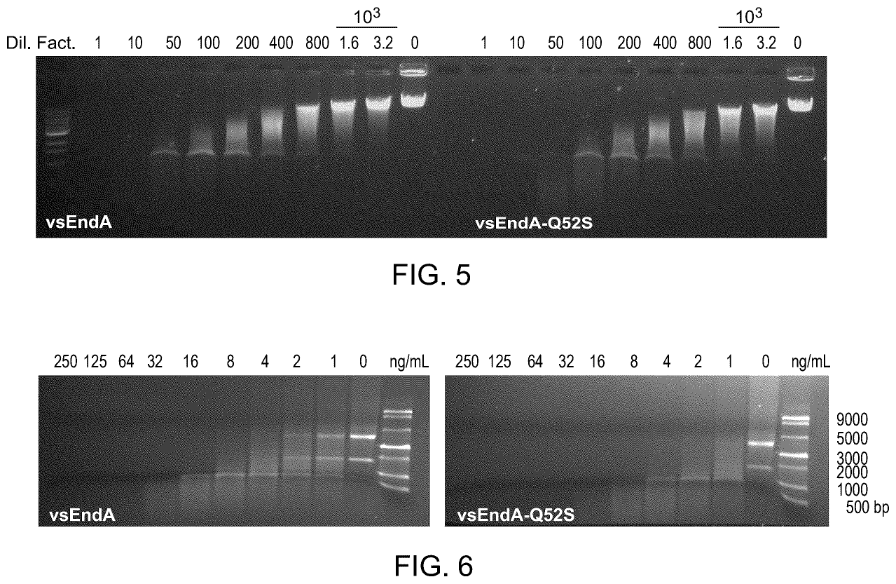

Serial dilutions of vsEndA and vsEndA-Q52S from 10 ng/ml to 3.125 μg/mL were prepared in 25 mM Tris-HCl pH 8.5, 0.5 M NaCl, 5 mM MgCl₂, 0.1 μg/mL BSA, 0.01% Triton reaction buffer containing 0.02 mg/mL of lambda DNA (New England Biolabs). The 20 μL reactions were incubated at 37° C. for 10 minutes and then stopped with 7 μL of loading buffer 6× (New England Biolabs). The final samples were loaded on a precast agarose gel 1.2% (Lonza) for analysis (FIG. 5).

When tested on lambda DNA at 37° C. for 10 min in a Tris buffer pH 8.5 with 500 mM NaCl, 0.05 ng of vsEndA digests 1 μg of lambda DNA (>95% digestion determined by visual observation on the agarose gel), while only 0.01 ng of vsEndA-Q52S is necessary (FIG. 5). These data show that vsEndA-Q52S is 5 times more active than vsEndA at digesting lambda DNA in the conditions tested.

### Example 6: Nuclease Activity on Total RNA from HEK Cells

Serial dilutions of vsEndA and vsEndA-Q52S were prepared in a 25 mM Tris-HCl pH 8.5, 500 mM NaCl, 5 mM MgCl₂, so that 250 ng/ml to 0.5 ng/ml of enzymes were mixed with 50 μg/mL of total RNA extract from HEK cells. The 20 μL reactions were incubated at 25° C. for 10 min, then stopped with 100 mM EDTA and mixed with 2×RNA loading buffer (New England Biolabs). 10 μL of the final samples were loaded on a precast agarose gel 1.2% (Lonza) for analysis (FIG. 6).

When tested on a total RNA extract from HEK cells at 25° C. for 10 min in a Tris buffer pH 8.5 with 500 mM NaCl, 3.1 ng of vsEndA digests 2.5 μg RNA, while 2 time less (1.6 ng) of vsEndA-Q52S is necessary (FIG. 6). These data indicate that vsEndA-Q52S is 2 times more performant than vsEndA at digesting extracted RNA when tested at 500 mM NaCl pH 8.5.

### Example 7: Nuclease Activity on a Fluorescent RNA Substrate

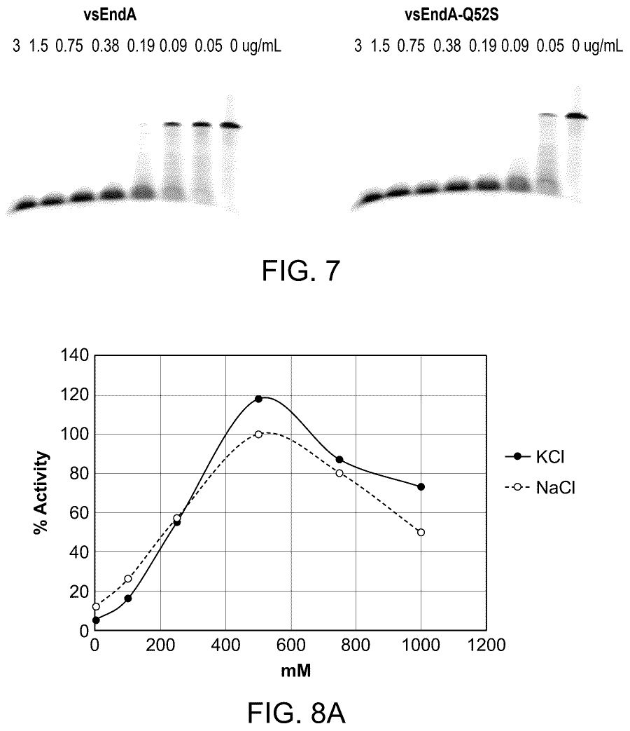

Serial dilutions of vsEndA and vsEndA-Q52S were prepared in a 25 mM Tris-HCl pH 8.5, 500 mM NaCl, 5 mM MgCl₂, 0.005% Tween 20 so that 3 μg/mL to 0.05 μg/mL of enzymes were mixed with 0.2 μM of FAM-RNA substrate (SEQ ID NO:9 with the 5′ FAM label), 0.8 μM unlabeled oligo (SEQ ID NO:9 without the 5′ FAM label) and 1 mg/mL of calf-thymus DNA in a total of 10 μL. The reactions were incubated at 25° C. for 10 min, then stopped with 10 μL of 2×RNA loading buffer (New England Biolabs). 12 μL of the final samples were loaded on a Novex™ TBE-Urea Gel 15% (ThermoFisher). The gel was analyzed on Typhoon instrument (Cytiva) using Cy2 setting (FIG. 7).

FIG. 7 shows that ~1.9 ng vsEndA-Q52S digests 0.2 μM of the fluorescent RNA substrate, which is 2 time less (3.8 ng) than vsEndA (FIG. 7). This show that vsEndA-Q52S is ~2 times more performant than vsEndA at digesting this fluorescent RNA substrate in the conditions tested.

### Example 8: Nuclease Activity in the Presence of KCl, Mg(NO₃)₂, or MgCl₂

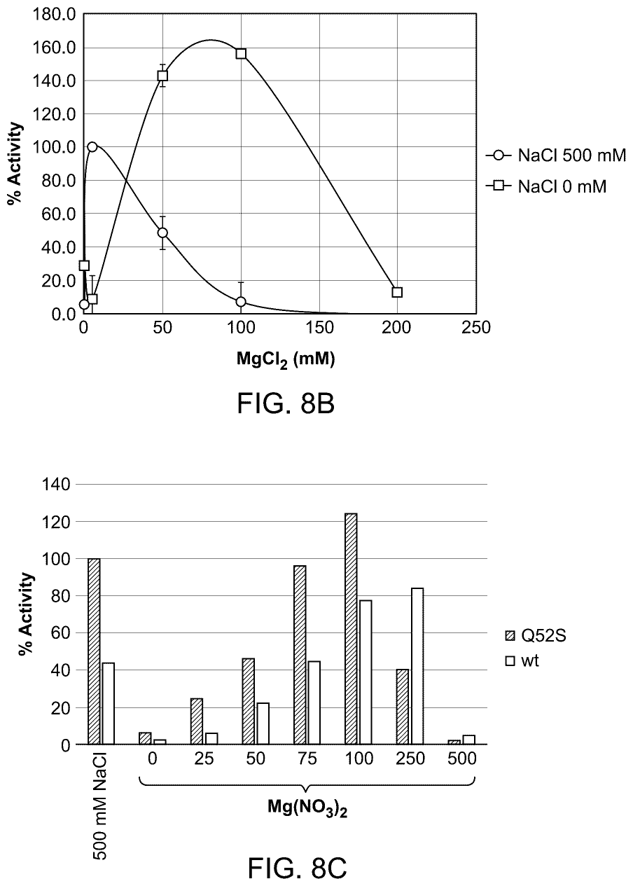

Nuclease activity was assessed in the presence of salts other than NaCl. The substitution of NaCl with KCl or Mg(NO₃)₂ was tested in 25 mM Tris-HCl pH 8.5, 5 mM MgCl₂ supplemented with 0 to 1000 mM KCl or 0 to 200 mM Mg(NO₃)₂, while the substitution of NaCl with MgCl₂ was tested in 25 mM Tris-HCl pH 8.5 with and without 500 mM NaCl supplemented with 0 to 200 mM MgCl₂. 0.1 μg/mL vsEndA-Q52S was mixed with 5 μM of the dsDNA substrate of Example 1 and 1 mg/mL of calf-thymus DNA in each buffer. Kinetic reactions were followed on a SpectraMax (Molecular Devices) at Ex 485 nm-Em 525 nm cutoff 515 nm every 30 sec for 10 min at 25° C. using 96 well plate half area black bottom. Values of released fluorescence in the linear range were chosen to compare each condition tested. The percentages of activity were calculated based on the highest value set at 100% (FIG. 8).

FIG. 8A shows that KCl can substitute NaCl at an equivalent concentration without impacting the enzyme activity. MgCl₂ can also replace NaCl when used between 25 to 150 mM, while in presence of 500 mM NaCl, MgCl₂ is required between 5 and 50 mM to maintain an activity >50% (FIG. 8B). In FIG. 8C, Mg(NO₃)₂ used between 75 mM to 250 mM can substitute 500 mM NaCl and provide >40% enzyme activity.

### Example 9: Nuclease Activity at High Substrate:Enzyme Ratios

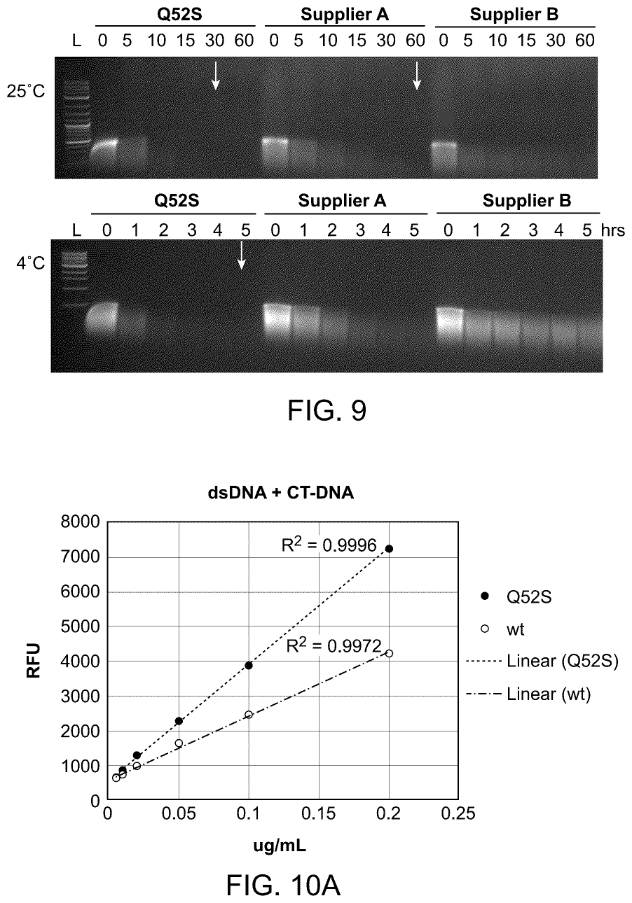

20 ng of vsEndA-Q52S, the endonuclease of Supplier A or the endonuclease of Supplier B was mixed with 150 μg of calf thymus DNA in their respective buffer (25 mM Tris-HCl pH 8.5, 5 mM MgCl₂ for vsEndA-Q52S and Supplier A, 50 mM Tris-HCl pH 8, 1 mM MgCl₂ for Supplier B) supplemented with 500 mM NaCl. The 150 μL reaction was incubated either at 25° C. or 4° C. 20 μL of the samples at 25° C. were harvested at 0, 5, 10, 15, 30, and 60 min, and every hour for the samples at 4° C., and mixed with 4 μL EDTA 500 mM (100 mM final) to stop the reaction. All samples were then mixed with loading buffer 6× (New England Biolabs) and loaded on a precast agarose gel 1.2% (Lonza) for analysis (FIG. 9).

FIG. 9 shows that 20 ng of vsEndA can digest 150 μg of calf thymus DNA in 30 min at 25° C. in a Tris buffer pH 8.5 with 500 mM NaCl. In comparison, the endonucleases supplier A and B needs twice (60 min) and more than 60 min, respectively. At 4° C., 20 ng of vsEndA can digest 150 μg of calf thymus DNA in about 5h, while the endonucleases of supplier A and B both need >5h.

### Example 10: Nuclease Activity at Various Substrate:Enzyme Ratios

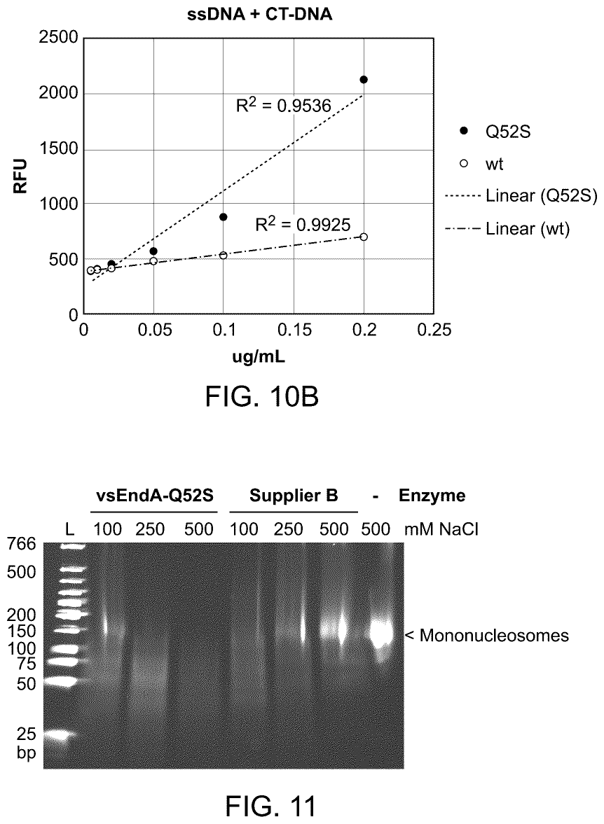

vsEndA and vsEndA-Q52S activity was tested on dsDNA and ssDNA fluorescent substrates at concentrations from 0.005 μg/mL to 0.2 μg/mL in 25 mM Tris-HCl pH 8.5, 500 mM NaCl, 5 mM MgCl₂ buffer in presence of 5 μM of the Example 1 dsDNA substrate or 5 μM of the Example 1 linear ssDNA substrate, and with 0.5 mg/mL of calf-thymus DNA. Each 50 μL reaction was loaded on a 96 well plate half area black bottom and incubated at 25° C. on a SpectraMax (Molecular Devices) to follow the kinetic at Ex 485 nm-Em 525 nm cutoff 515 nm every 10 sec for 5 min. The fluorescence released (in RFU) in the linear range for all conditions were plotted versus enzyme concentration. Linear trendline and R-squared values are displayed on both graphs (FIG. 10).

FIG. 10 shows the linear response for each enzyme on the fluorescence released from ssDNA and dsDNA substrates as they are degraded versus their concentration. vsEndA-Q52S is about 2× more active on dsDNA and 4× on ssDNA compared to vsEndA.

### Example 11: Nuclease Activity on Mononucleosomes

Nuclease activity of vsEndA-Q52S and (for comparison) the Supplier B enzymes were tested on mononucleosomes. Enzymes were diluted at 1 μg/mL in their respective buffer (vsEndA-Q52S: 25 mM Tris-HCl pH 8.5, 5 mM MgCl₂, Supplier B: 25 mM Tris-HCl pH 8, 1 mM MgCl₂) supplemented with 100, 250, 500 mM NaCl. Two microliters (2 ng) of each enzyme were mixed with 2 μg of HeLa purified mononucleosomes (EpiCypher), in a 30 μL reaction using each respective buffer. After an incubation at 20° C. for 5 min, all reactions were stopped with 6 μL of EDTA 500 mM. All samples were then mixed with loading buffer 6× (New England Biolabs) and loaded on a precast 20% TBE gel (Invitrogen) for analysis (FIG. 11).

FIG. 11 shows that the mononucleosomal DNA, resolving at 150 pb (marked), is progressively degraded with vsEndA-Q52S as the concentration of NaCl increases, to reach almost full digestion at 500 mM NaCl. On the other hand, the mononucleosomal DNA is more resistant to degradation with the endonuclease of Supplier B at the 3 NaCl concentrations tested. Nucleosomal DNA may dissociate from nucleosomal proteins (e.g., histones) with increasing salt concentrations. Without limiting any embodiment to any particular mechanism of action, at lower salt concentrations the association of nucleosomal DNA and histones may influence or limit enzyme-DNA interaction (visible DNA smears at 100 and 250 mM for both enzymes) whereas the DNA may be more accessible at high salt (500 mM) but only vsEndA-Q52S retained sufficient activity to digest DNA under these conditions.

What is claimed is:

1. A salt active nuclease variant having an amino acid sequence that is at least 90% identical to one or more of SEQ ID NOS:1-4, having at its position corresponding to position 52 of SEQ ID NO:1 an amino acid other than Q, and having catalytic activity as an endonuclease and/or an endoribonuclease at a total salt concentration of 250 mM to 1 M.

2. A salt active nuclease variant according to claim 1, wherein the amino acid sequence is identical to SEQ ID NO:1 at its position corresponding to S52 of SEQ ID NO:1.

3. A salt active nuclease variant according to claim 1, wherein the amino acid sequence that is at least 95% identical to one or more of SEQ ID NOS:1-4.

4. A salt active nuclease variant according to claim 1, wherein the amino acid sequence is identical to SEQ ID NO:2.

5. A salt active nuclease variant according to claim 1, wherein the amino acid sequence is identical to SEQ ID NO:3, wherein (a) X1 is a signal peptide, a purification tag, or a linker and X215 is any amino acid or absent, or (b) X1 is any amino acid or absent and X215 is a purification tag or a linker or X215.

6. A salt active nuclease variant according to claim 1, wherein all substitutions are conservative substitutions.

7. A salt active nuclease variant according to claim 1, wherein all substitutions are conservative substitutions except one, which is a non-conservative substitution.

8. A salt active nuclease variant according to claim 1, wherein all substitutions are conservative substitutions except two, which are independent non-conservative substitutions.

9. A salt active nuclease variant according to claim 1, wherein the amino acid sequence is at least 98% identical to any of SEQ ID NOS:1-4.

10. A salt active nuclease variant according to claim 9, wherein the amino acid sequence is at least 98% identical to SEQ ID NO:2 and wherein X47-X51 and X53-X56 are each, independently, any amino acid.

11. A salt active nuclease variant according to claim 9, wherein the amino acid sequence is at least 98% identical to any of SEQ ID NO:2 and wherein:
(a) X47, X49-X51, and X53-X55 are each, independently, C, D, E, H, K, N, P, Q, R, S, T, or Y, and
(b) X48 and X56 are each, independently, A, L, I, M, W, F, C, G, or P.

12. A salt active nuclease variant according to claim 1, wherein the salt active nuclease variant has at least 30% of its peak catalytic activity in the presence of a total salt concentration of 250 mM to 1 M, the salt consisting essentially of NaCl, KCl, or NaCl and KCl.

13. A salt active nuclease variant according to claim 1, wherein the salt active nuclease variant has at least 50% of its peak catalytic activity in the presence of a total salt concentration of 250 mM to 750 mM, the salt consisting essentially of NaCl, KCl, or NaCl and KCl.

14. A salt active nuclease variant according to claim 1, wherein the salt active nuclease variant has at least 70% of its peak catalytic activity in the presence of a total salt concentration of 300 mM to 600 mM, the salt consisting essentially of NaCl, KCl, or NaCl and KCl.

15. A salt active nuclease variant according to claim 1, wherein the salt active nuclease variant has at least 30% of its peak catalytic activity in the presence of a total salt concentration of 50 mM to 150 mM, the salt consisting essentially of MgCl₂, Mg(NO₃)₂, or MgCl₂ and Mg(NO₃)₂.

16. A salt active nuclease variant according to claim 1, wherein the salt active nuclease variant has at least 70% of its peak catalytic activity at pH ≥8.

17. A salt active nuclease variant according to claim 1, wherein the salt active nuclease variant has at least 70% of its peak catalytic activity at 40° C.-55° C.

18. A salt active nuclease variant according to claim 1, wherein the salt active nuclease variant is an immobilized salt active nuclease variant comprising the salt active nuclease variant and a solid support.

19. A salt active nuclease variant according to claim 18, wherein the solid support is a magnetic bead, an agarose bead, a polystyrene bead, a polyacrylamide bead or a chitin bead.

20. A composition comprising:
(a) a salt active nuclease variant according to claim 1; and
(b) 250 mM to 1 M salt.

21. A composition according to claim 20 further comprising a buffering agent.

22. A kit comprising:
(a) a salt active nuclease variant according to claim 1; and
(b) a buffering agent.

23. A fusion protein comprising a single polypeptide chain, the single peptide chain comprising:
(a) a salt active nuclease variant according to claim 1; and
(b) a SSO7d DNA binding peptide, a transcription factor, an antibody, protein A, a maltose binding domain, a histidine tag, a chitin binding domain, an alpha mating factor, an O⁶-alkylguanine-DNA alkyltransferase, and/or albumin.

24. A method for hydrolyzing DNA and RNA comprising contacting (a) a composition comprising DNA and RNA, and (b) a salt active nuclease variant according to claim 1 to form a reaction mixture comprising DNA hydrolysis products and RNA hydrolysis products.

25. A method according to claim 24, wherein the composition comprising DNA and RNA and/or the reaction mixture further comprises a total salt concentration of 250 mM to 1 M, the salt consisting essentially of NaCl, KCl, or NaCl and KCl.

26. A method according to claim 24, wherein the composition comprising DNA and RNA and/or the reaction mixture further comprises a total salt concentration of 50 mM to 150 mM, the salt consisting essentially of MgCl₂, Mg(NO₃)₂, or MgCl₂ and Mg(NO₃)₂.

27. A method according to claim 24, wherein the reaction mixture comprises ≤10% of the DNA that was in the composition.

28. A method according to claim 24, wherein the reaction mixture comprises ≤10% of the RNA that was in the composition.

29. A method according to claim 24, wherein the composition comprising DNA and RNA further comprises one or more DNA binding proteins.

30. A method according to claim 24, wherein the composition comprising DNA and RNA further comprises nucleosomes.


SEQUENCE LISTING (9 sequences)

SEQ ID NO: 1 - protein, 213 aa, synthetic construct
APPSSFSKAKKEAVKIYLDYPTSFYCGCDITWKNKKKGIPELESCGYQVRKSEKRASRIEWEHVVPAWQFGHQRQCWQKGGRKNCTRNDKQFKSMEADLHNLVPAIGEVNGDRSNFRFSQWNGSKGAFYGQCAFKVDFKGRVAEPPAQSRGAIARTYLYMNNEYKFNLSKAQRQLMEAWNKQYPVSTWECTRDERIAKIQGNHNQFVYKACTK

SEQ ID NO: 2 - protein, 213 aa, synthetic construct
  VARIANT 47: note = X may be any amino acid (e.g., one of C, D, E, H, K, N, P, Q, R, S, T, and Y)
  VARIANT 49: note = X may be any amino acid (e.g., one of C, D, E, H, K, N, P, Q, R, S, T, and Y)
  VARIANT 50: note = X may be any amino acid (e.g., one of C, D, E, H, K, N, P, Q, R, S, T, and Y)
  VARIANT 51: note = X may be any amino acid (e.g., one of C, D, E, H, K, N, P, Q, R, S, T, and Y)
  VARIANT 53: note = X may be any amino acid (e.g., one of C, D, E, H, K, N, P, Q, R, S, T, and Y)
  VARIANT 54: note = X may be any amino acid (e.g., one of C, D, E, H, K, N, P, Q, R, S, T, and Y)
  VARIANT 55: note = X may be any amino acid (e.g., one of C, D, E, H, K, N, P, Q, R, S, T, and Y)
  VARIANT 52: note = X is any amino acid other than Q (e.g., C, D, E, H, K, P, R, S, T or Y)
  VARIANT 48: note = X may be any amino acid (e.g., one of A, L, I, M, W, F, C, G, and P)
  VARIANT 56: note = X may be any amino acid (e.g., one of A, L, I, M, W, F, C, G, and P)
  VARIANT 1: note = X may be a purification tag, a linker, any amino acid or may be absent
APPSSFSKAKKEAVKIYLDYPTSFYCGCDITWKNKKKGIPELESCGYXXXXXXXXXSRIEWEHVVPAWQFGHQRQCWQKGGRKNCTRNDKQFKSMEADLHNLVPAIGEVNGDRSNFRFSQWNGSKGAFYGQCAFKVDFKGRVAEPPAQSRGAIARTYLYMNNEYKFNLSKAQRQLMEAWNKQYPVSTWECTRDERIAKIQGNHNQFVYKACTK

SEQ ID NO: 3 - protein, 215 aa, synthetic construct
  VARIANT 1: note = X may be a purification tag, a linker, any amino acid or may be absent
  VARIANT 53: note = X is any amino acid other than Q (e.g., C, D, E, H, K, N, P, R, S, T or Y)
  VARIANT 215: note = X may be a signal peptide, a purification tag, a linker, any amino acid or may be absent
XAPPSSFSKAKKEAVKIYLDYPTSFYCGCDITWKNKKKGIPELESCGYQVRKXEKRASRIEWEHVVPAWQFGHQRQCWQKGGRKNCTRNDKQFKSMEADLHNLVPAIGEVNGDRSNFRFSQWNGSKGAFYGQCAFKVDFKGRVAEPPAQSRGAIARTYLYMNNEYKFNLSKAQRQLMEAWNKQYPVSTWECTRDERIAKIQGNHNQFVYKACTKX

SEQ ID NO: 4 - protein, 234 aa, synthetic construct
MKLIRLVISLIAVSFTVNVMAAPPSSFSKAKKEAVKIYLDYPTSFYCGCDITWKNKKKGIPELESCGYQVRKSEKRASRIEWEHVVPAWQFGHQRQCWQKGGRKNCTRNDKQFKSMEADLHNLVPAIGEVNGDRSNFRFSQWNGSKGAFYGQCAFKVDFKGRVAEPPAQSRGAIARTYLYMNNEYKFNLSKAQRQLMEAWNKQYPVSTWECTRDERIAKIQGNHNQFVYKACTK

SEQ ID NO: 5 - protein, 234 aa, synthetic construct
MKLIRLVISLIAVSFTVNVMAAPPSSFSKAKKEAVKIYLDYPTSFYCGCDITWKNKKKGIPELESCGYQVRKQEKRASRIEWEHVVPAWQFGHQRQCWQKGGRKNCTRNDKQFKSMEADLHNLVPAIGEVNGDRSNFRFSQWNGSKGAFYGQCAFKVDFKGRVAEPPAQSRGAIARTYLYMNNEYKFNLSKAQRQLMEAWNKQYPVSTWECTRDERIAKIQGNHNQFVYKACTK

SEQ ID NO: 6 - protein, 213 aa, synthetic construct
APPSSFSAAKQQAVKIYQDHPISFYCGCDIEWQGKKGIPNLETCGYQVRKQQTRASRIEWEHVVPAWQFGHHRQCWQKGGRKNCSKNDQQFRLMEADLHNLTPAIGEVNGDRSNFNFSQWNGVDGVSYGRCEMQVNFKQRKVMPQTELRGSIARTYLYMSQEYGFQLSKQQQQLMQAWNKSYPVDEWECTRDDRIAKIQGNHNPFVQQSCQTQ

SEQ ID NO: 7 - DNA, 35 bp, synthetic construct
  source 1..35: mol_type = other DNA
tctaagccgtgtacattttttgtacacggcttaga

SEQ ID NO: 8 - DNA, 15 bp, synthetic construct
  source 1..15: mol_type = other DNA
tgaagtaatctgtta

SEQ ID NO: 9 - RNA, 45 nt, synthetic construct
  source 1..45: mol_type = other RNA
aaggagaagagaagaggaagaaaactaacacaggagagagaagga